(19) United States
(12) Patent Application Publication
(10) Pub. No.: US 2008/0011052 A1
(43) Pub. Date: Jan. 17, 2008

(54) SOOT SENSOR

(76) Inventors: Tomonori KONDO, Komaki-shi (JP); Norihiko NADANAMI, Inuyama-shi (JP); Daisuke KOMATSU, Nisshin-shi (JP); Hitoshi YOKOI, Ama-gun (JP); Masato KATSUTA, Komaki-shi (JP)

Correspondence Address: STITES & HARBISON PLLC, 1199 NORTH FAIRFAX STREET, SUITE 900, ALEXANDRIA, VA, 22314, US

(21) Appl. No.: 11/770,396

(22) Filed: Jun. 28, 2007

(30) Foreign Application Priority Data
Jul. 3, 2006 (JP) P.2006-182915
May 8, 2007 (JP) P.2007-123035

(51) Int. Cl. G01N 33/00

(52) U.S. Cl. 73/23.31

(57) ABSTRACT

A soot sensor includes a center electrode extending in an axial direction and a cylindrical insulator from which a leading end of the center electrode protrudes. The insulator is provided around a periphery of the center electrode and includes a heating element. The soot sensor also includes a sealing member sealing a gap between the insulator and the center electrode.

### BACKGROUND OF THE INVENTION

1. Field of the Invention

The present invention relates to a soot sensor.

2. Description of the Related Art

Conventionally, a detecting portion provided in a smoke detecting device such as that disclosed in JP-U-64-50355 is referred to as a soot sensor. The detecting portion of this type of smoke detecting device has a rod-like center electrode accommodated in a metal shell through an insulator, and a leading end of the center electrode extends outwardly from the insulator so as to be exposed to the outside. In addition, an outer electrode joined to the metal shell is disposed with a gap with respect to the leading end of the center electrode. A spark discharge is generated when a high voltage is applied across the center electrode and the outer electrode under conditions in which the center electrode and the outer electrode are exposed to exhaust gas. At this time, the presence of soot in the exhaust gas and the amount of this soot are detected based on the discharge voltage by making use of the principle that the greater the increase in the amount of soot in the exhaust gas, the greater the reduction in the voltage (corresponding to the discharge voltage) at the time of the occurrence of the spark discharge.

In the detecting portion constructed as described above, if the soot is attached to the insulator, soot detection accuracy declines. Further, in removing the soot thus attached, the spark discharge is insufficient, and thus it is desirable to eliminate the soot by use of a heating element.

For this reason, if a heating element as described in W. D. E. Allan, R. D. Freeman, G. R. Pucher, D. Faux and M. F. Bardon, “DEVELOPMENT OF A SMOKE SENSOR FOR DIESEL ENGINES, Royal Military College of Canada, D. P. Gardiner, Nexum Research Corporation, p. 220, Powertrain & Fluid Systems Conference, Oct. 27-30, 2003 is provided for the aforementioned detecting portion, it is possible to eliminate the soot attached to the center electrode and the outer electrode.

However, if the heating element is provided for the detecting portion as described above, the discharge voltage declines even in a gas atmosphere where there is practically no soot. Even if the spark discharge is effected under this condition by exposing the center electrode and the outer electrode to the exhaust gas containing soot, the discharge voltage does not significantly decline and thus does not accurately reflect the presence of soot. For this reason, it is difficult to detect from the discharge voltage the presence of soot and the amount of soot.

Considering this point in more detail, since the soot is a collection of electrically conductive particles which are carbon particles, the soot itself is a cause of the aforementioned decline in the discharge voltage. On the other hand, given the fact that the discharge voltage will decline even in a gas atmosphere where there is practically no soot as described above, it is conceivable that, in addition to soot, particles are present which contribute to electrical conductivity, such as ions exhibiting substantially similar activity to that of the soot.

### SUMMARY OF THE INVENTION

Accordingly, the present invention is based, at least in part, on the above-described considerations, and one object of the invention is to provide a soot sensor which includes a cylindrical insulator including a heating element as well as a center electrode protruding from a leading end of the insulator, and which is arranged to effect an electric discharge without being affected by particles, other than soot, contributing to electrical conductivity.

To attain the foregoing object and other objects, in accordance with a first aspect of the invention there is provided a soot sensor including:

a center electrode (which, for example, is advantageously of a rod-like configuration) extending in an axial direction; and

a cylindrical insulator, provided around a periphery of the center electrode, from which a leading end of the center electrode protrudes, the insulator including a heating element; and

a sealing member which seals a gap between the insulator and the center electrode.

According to the above-described first aspect of the invention, the gap between the insulator and the center electrode is sealed with a sealing member and for this reason, when a high voltage is applied to the center electrode, the high voltage is also applied across the heating element and the center electrode. As a result, because an electrical discharge occurs between the heating element and the center electrode, particles which contribute to electrical conductivity, such as ions, are generated between the insulator and the center electrode. However, these particles are sealed within the insulator by the sealing member, and cannot move to the discharge portion.

Accordingly, the discharge voltage of the above-described discharge portion declines only because of the presence of soot, without being affected by the aforementioned particles contributing to electrical conductivity. As a result, according to the soot sensor of the invention, soot can be detected with high accuracy without being affected by the particles contributing to electrical conductivity.

In accordance with a second aspect of the invention, in the soot sensor according to the first aspect of the invention, the sealing member is provided on a leading end of the insulator so as to cover the gap.

Because the sealing member is thus provided on the leading end of the insulator so as to cover the gap, it is possible to seal the gap between the insulator and the center electrode. Thus, according to this aspect of the invention, soot can be detected with high accuracy without being affected by particles which contribute to electrical conductivity.

In accordance with a third aspect of the invention, in the soot sensor according to the second aspect of the invention, the sealing member is formed of at least one of a glass and a ceramic.

According to the above-described third aspect of the invention, because the sealing member is formed of a glass, ceramic or both, the sealing member is not only compact but also is heat resistant. Accordingly, the sealing member is capable of properly sealing the gap between the insulator and the center electrode even under the high heating temperatures associated with the heating element.

In accordance with a fourth aspect of the invention, in the soot sensor according to the third aspect of the invention, the leading end of the heating element and the leading end of the sealing member are spaced apart by a distance between 3 mm and 12 mm along an outer surface of the insulator.

Thus, because the lower limit of the distance or spacing between the leading end of the heating element and the leading end of the sealing member along the outer surface is 3 mm, it can be ensured that the heating element is not located too close to the leading end of the center electrode. Accordingly, it is possible to prevent the heating element from forming a short-circuit with the center electrode or generating a discharge. In addition, because the upper limit of the spacing or distance between the leading end of the heating element and the leading end of the sealing member along the outer surface is 12 mm, it is possible to prevent the soot from becoming deposited on the insulator and the sealing member.

In accordance with a fifth aspect of the invention, in the soot sensor according to the third or fourth aspect of the invention, the soot sensor further comprises: a hollow metal shell provided around a periphery of the insulator, wherein the leading end of the sealing member is located closer to a rear end side of the sensor than the leading end of the metal shell.

Thus, because the leading end of the sealing member is located closer to the rear end side of the sensor than the leading end of the metal shell, it is more difficult for the soot to reach to the insulator or the sealing member from outside the metal shell, thereby making it possible to prevent the soot from being deposited on the insulator or the sealing member.

In accordance with a sixth aspect of the invention, in the soot sensor according to the second aspect of the invention, the sealing member is formed of a metal.

According to the above-described sixth aspect of the invention, the sealing member is not only compact or dense but is also heat resistant. Accordingly, the sealing member is capable of properly sealing the gap between the insulator and the center electrode even under the heating temperatures associated with the heating element.

In accordance with a seventh aspect of the invention, in the soot sensor according to the first aspect of the invention, the sealing member is provided in the gap at a position closer to a leading end side of the sensor than at least the heating element.

Thus, by providing the sealing member in the gap at a position closer to the leading end side of the sensor than at least the heating element, it is possible to suitably seal the gap between the insulator and the center electrode. According to this aspect of the invention, soot can be detected with high accuracy without being affected by particles which contribute to electrical conductivity.

In accordance with an eighth aspect of the invention, in the soot sensor according to the seventh aspect of the invention, the sealing member is formed of at least one of a glass, a ceramic, and a metal.

According to the above-described eighth aspect of the invention, the sealing member is not only compact but is also heat resistant. Consequently, the sealing member is capable of properly sealing the gap between the insulator and the center electrode even under the heating temperatures associated with the heating element.

In accordance with a ninth aspect of the invention, in the soot sensor according to any one of the sixth to eighth aspects of the invention, the distance or spacing between the leading end of the heating element and the leading end of the insulator along an outer surface of the insulator is between 3 mm and 12 mm, i.e., not less than 3 mm and not more than 12 mm.

When the lower limit of the distance between the leading end of the heating element and the leading end of the insulator along the outer surface is set to 3 mm, the heating element is not located too close to the leading end of the sealing member or the center electrode. Accordingly, it is possible to prevent the heating element from short-circuiting with the sealing member or the center electrode or generating a discharge. In addition, when the upper limit of the distance between the leading end of the heating element and the leading end of the insulator along the outer surface is set to 12 mm, it is possible to prevent the soot from becoming deposited on the insulator.

In accordance with a tenth aspect of the invention, in the soot sensor according to any one of the sixth to ninth aspects of the invention, the soot sensor further comprises: a hollow metal shell provided around a periphery of the insulator, wherein the leading end of the insulator is located closer to a rear end side of the sensor than the leading end of the metal shell.

Because the leading end of the insulator is located closer to the rear end side of the sensor than the leading end of the metal shell, the soot is unlikely to reach the insulator from outside the metal shell, thereby making it possible to prevent the soot from being deposited on the insulator.

In accordance with an eleventh aspect of the invention, in the soot sensor according to any one of the first to tenth aspects of the invention, the center electrode is a positive side or positive electrode.

According to the above-described eleventh aspect of the invention, because the center electrode is a positive side electrode, although particles contributing to electrical conductivity (conductive particles), such as ions, are likely to be generated in the gap between the insulator and the center electrode, by using the soot sensor of the invention, any decline in the discharge voltage is only caused by soot, without being affected by the conductive particles. As a consequence, according to the soot sensor of the invention, the soot can be detected with a high degree of accuracy without being affected by the conductive particles.

In accordance with a twelfth aspect of the invention, in the soot sensor according to any one of the first to eleventh aspects of the invention, the insulator has a thickness of 0.7 mm to 3 mm at the position at which the heating element is disposed.

Because the insulator thus has a thickness of not less than 0.7 mm at the position at which the heating element is disposed, it is possible to prevent a voltage discharge from taking place in the “thicknesswise” or transverse direction of the insulator which would otherwise occur because the insulator is too thin. Because the insulator has a thickness of not more than 3 mm at the position at which the heating element is disposed, it is possible to prevent an increase in heat capacity which would otherwise occur due to the fact that the insulator is too thick.

Further features and advantages of the present invention will be set forth in, or apparent from, the detailed description of preferred embodiments thereof which follows.

### BRIEF DESCRIPTION OF THE DRAWINGS

FIG. 1 is a fragmentary side elevational view illustrating a first embodiment of a spark plug type soot sensor in accordance with the invention;

FIG. 2 is a cross-sectional view taken along line 2-2 in FIG. 1;

FIG. 3 is an enlarged fragmentary plan view of a heater of the first embodiment;

FIG. 4 is a fragmentary side elevational view illustrating a second embodiment of a spark plug type soot sensor in accordance with the invention;

FIG. 5 is a cross-sectional view taken along line 6-6 in FIG. 4;

FIG. 6 is a fragmentary plan view illustrating selected portions of a soot sensor in accordance with the a third embodiment of the invention;

FIG. 7 is a fragmentary side elevational view illustrating a fourth embodiment of the invention; and

FIG. 8 is a fragmentary side elevational view illustrating a fifth embodiment of the invention.

### DETAILED DESCRIPTION OF THE INVENTION

Hereafter, a description will be given of embodiments of the invention with reference to the drawings.

### First Embodiment

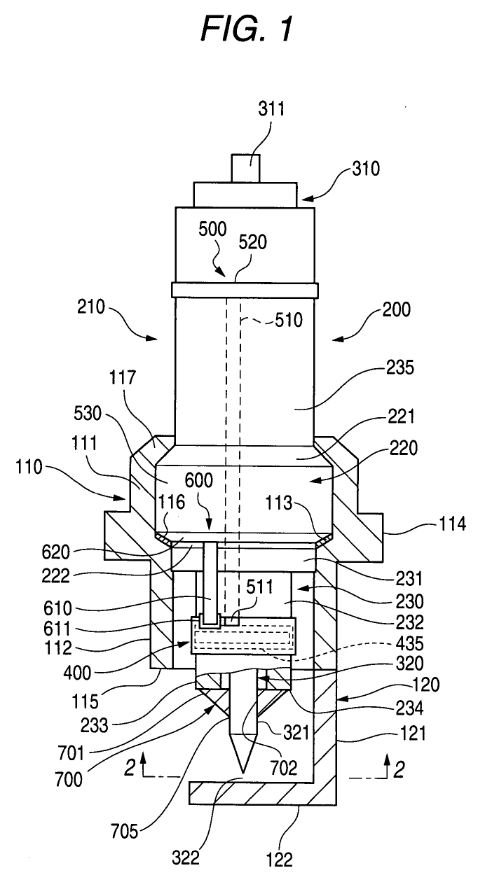

FIG. 1 shows of a spark plug type soot sensor in accordance with a first embodiment of the invention. This soot sensor is basically comprised of a metal shell 110, an insulator 200, and a center electrode 320.

The metal shell 110 is preferably formed of a soft steel and has a base end portion 111, a leading end portion 112, and a collar portion 114 connecting the base end portion 111 and the leading end portion 112.

The leading end portion 112 has a smaller inside diameter than the base end portion 111. In addition, the collar portion 114 has on its inner peripheral surface an inclined portion 113 which is inclined inwardly from the base end portion 111 toward the leading end portion 112, i.e., is of an inwardly tapered form beginning at the base end portion 111.

An outer electrode 120 is fixed to a leading end 115 of the metal shell 110. This outer electrode 120 has a connecting portion 121 and an electrode portion 122. The connecting portion 121 is connected to the leading end 115 of the metal shell 110 and extends in parallel to the vertical axis of the leading end portion 112.

The electrode portion 122 extends from the connecting portion 121 in the radial-direction of the metal shell 110 and is positioned opposite to the center electrode 320, which will be described later. It should be noted that in this first embodiment, the outer electrode 120 is used as a negative electrode. In addition, in making the outer electrode 120, a material which is typically used for a spark plug, such as a nickel alloy, iridium, platinum, tungsten, or SUS steel, is used.

The insulator 200 is formed of a ceramic and has a base end portion 210, an intermediate portion 220, and a leading end portion 230.

The intermediate portion 220 is formed so as to have a larger outside diameter than the base end portion 210 and the leading end portion 230. For this reason, the outer peripheral surface of the intermediate portion 220 at its both axial end portions forms or constitutes inclined or tapered portions 221 and 222 which are, respectively, inclined inwardly toward (i) the outer peripheral surface of the base end portion 210 and (ii) the outer peripheral surface of a large-diameter portion 231 (which will be described later) of the leading end portion 230.

As shown in FIG. 1, the leading end portion 230 is constituted by the large-diameter portion 231 and a small-diameter portion 232 which are formed concentrically with each other. It should be noted that, in this embodiment, the small-diameter portion 232 is formed in such a manner as to be slightly inclined or tapered from its end adjacent the large-diameter portion 231 toward its leading end.

In the insulator 200 constructed as described above, its leading end portion 230 is inserted in the leading end portion 112 of the metal shell 110, and the large-diameter portion 231 is fitted in the leading end portion 112 of the metal shell 110. In addition, the intermediate portion 220 of the insulator 200 is fitted in the base end portion 111 and the collar portion 114 of the metal shell 110, and the inclined portion 222 is retained on the inclined portion 113 of the leading end portion 112 by means of packing 116. As a result, the insulator 200 is coaxially supported in the metal shell 110. It should be noted that an opening portion 117 of the base end portion 111 of the metal shell 110 is preferably engaged with the inclined portion 221 of the intermediate portion 220 of the insulator 200 by caulking.

The center electrode 320 is connected at its base end 311 to a high-voltage circuit (not shown), and a conducting member 310 is formed in such a manner as to cover a peripheral portion of the base end 311.

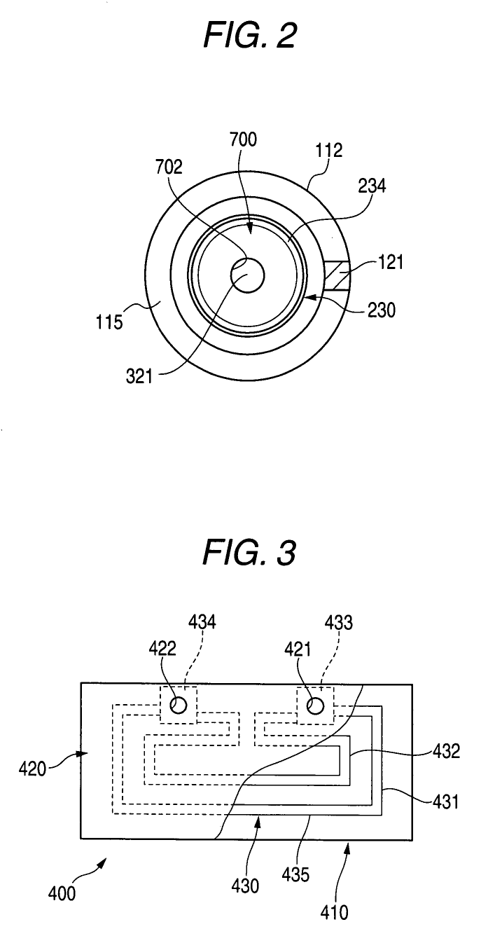

As can be seen from FIGS. 1 and 2, the center electrode 320 extends from the leading end portion 230 of the insulator 200 toward the electrode portion 122 of the outer electrode 120. In addition, a gap 233 is formed between the outer peripheral surface of the center electrode 320 and the inner peripheral surface of the cylindrical member 200.

The center electrode 320 has a leading end 321 protruding from the leading end of the insulator 200, and the leading end 321 is disposed opposite to the electrode portion 122 of the outer electrode 120 and spaced therefrom by a discharge gap 322 of, in this embodiment, 0.5 mm.

It should be noted that the tip of the leading end 321 of the center electrode 320 is tapered, and, in this embodiment, the apex angle formed is 60 degrees. In addition, the outside diameter (excluding the tapered portion at the tip) of the leading end portion 321 of the center electrode 320 is, in this embodiment, 2 mm. The center electrode 320 is used as a positive electrode.

In the soot sensor in accordance with this first embodiment, when a high voltage is applied across the outer electrode 120 and the center electrode 320 from the high-voltage circuit, the outer electrode 120 and the center electrode 320 discharges between the electrode portion 122 and the leading end 321 opposing each other. At this time, the voltage applied across the electrode portion 122 and the leading end 321 is detected as the voltage at the time of discharge (hereafter also referred to as the discharge voltage). It should be noted that, as discussed above, this discharge voltage declines when soot is present between the electrode portion 122 and the leading end 321.

In this first embodiment, the high voltage is set to a voltage of, for example, 10 kV for allowing a discharge to take place between the electrode portion 122 and the leading end 321 by dielectrically breaking down the air between the electrode portion 122 and the leading end 321 on the precondition of the aforementioned discharge gap 322.

As shown in FIG. 1, the insulator 200 of the soot sensor of this first embodiment has a heater 400 which extends around the entire periphery of an outer surface 235 of the small-diameter portion 232 of insulator 200.

Heater 400 effects heat cleaning of the electrode portion 122 and the leading end 321 by heating the insulator 200 and functions to prevent potential short-circuiting due to the soot deposited on the electrode portion 122 and the leading end 321.

As shown in FIG. 3, heater 400 includes two alumina sheets 410 and 420 and a heating element 430. The heating element 430 includes a strip-shaped outer heating resistor portion 431, a strip-shaped inner heating resistor portion 432, and positive and negative “both side” electrode pads 433 and 434. The heating resistor portions 431 and 432 and the electrode pads 433 and 434 are respectively formed by print-baking a platinum paste on the alumina sheet 410 with a pattern such as the one shown in FIG. 3.

In addition, the positive-side electrode pad 433 is connected to respective one-side end portions of (i) the outer heating resistor portion 431 and (ii) the inner heating resistor portion 432, and functions as a positive-side connecting terminal of the heater 400. Similarly, the negative-side electrode pad 434 is connected to respective other-side end portions of (i) the outer heating resistor portion 431 and (ii) the inner heating resistor portion 432, and functions as a negative-side connecting terminal of the heater 400.

The alumina sheet 420 is pressure-bonded to an inner surface of the alumina sheet 410 with the heating element 430 placed therebetween. This alumina sheet 420 includes through holes 421 and 422. The through hole 421 is located in correspondence with, i.e., in alignment with, a central portion of the positive-side electrode pad 433, while the through hole 422 is located in correspondence with a central portion of the negative-side electrode pad 434.

In the heater 400 thus constructed, when soot has been deposited on the insulator 200 to such an extent as to hamper proper discharge between the electrode portion 122 and the leading end 321, the heating element 430 begins heating in response to the application thereto of a heater voltage (of, e.g., 15 V) from a heater driving circuit (not shown), and performs heat cleaning. It should be noted that this heat cleaning is carried out under conditions in which the application of a high voltage from the aforementioned high-voltage circuit (not shown) across the electrode portion 122 and the leading end 321 is terminated.

In addition, as shown in FIG. 1, the soot sensor in accordance with this first embodiment has positive and negative “both-side” leads 500 and 600 for the heater 400, as well as a glass film 530 for these positive and negative both-side leads 500 and 600.

The positive-side lead 500 has an axial lead portion 510 and a circumferential lead portion 520. The axial lead portion 510 is provided on the insulator 200 so as to extend in the axial direction (see FIG. 1), and a leading end 511 of the axial lead portion 510 is provided on the electrode pad 433 (see FIG. 3) of the heater 400.

In addition, the circumferential lead portion 520 is provided over the entire periphery of the base end portion 210 of the insulator 200.

The negative-side lead 600 is provided on the insulator 200 through the glass film 530, and this negative-side lead 600 has an axial lead portion 610 and a circumferential lead portion 620.

The axial lead portion 610 has its leading end 611 disposed on the electrode pad 434 of the heater 400, and extends in the axial direction of the leading end portion 230 of the insulator 200 (see FIG. 1).

The circumferential lead portion 620 extends circumferentially around the inclined portion 222 of the intermediate portion 220 of the insulator 200. It should be noted that the circumferential lead portion 620 is provided separately from the axial lead portion 510 through the glass film 530 which will be described below.

The glass film 530 is provided over the entire periphery of the outer surface 235 of the insulator 200 in such a manner as to extend from a rear end of the heater 400 to the base end portion 210 through the intermediate portion 220 so as to cover the axial lead portion 510 (excluding the leading end 511).

In addition, the soot sensor in accordance with this first embodiment has a seal member 700, as shown in FIGS. 1 and 2. This seal member 700 is formed of a below-described sealing material and has the cross-sectional shape of a hollow truncated cone. Member 700 abuts against an outer peripheral surface of the leading end 321 of the center electrode 320 and a leading end 234 of the leading end portion 230 of the insulator 200. Further, a bottom surface 701 of the seal member 700 and the outer peripheral surface of the leading end 321 of the center electrode 320, as well as an inner peripheral surface 702 of the seal member 700 and the leading end 234 of the leading end portion 230 of the insulator 200, are in close, air-tight contact with each other.

In the first embodiment, the seal member 700 is formed as follows: First, a glass powder (made by Asahi Glass Co., Ltd., for example) whose principal components are SiO₂, B₂O₃, and ZnO is prepared as the aforementioned sealing material. This glass powder is formed into a paste so as to produce a glass powder paste. This glass powder paste is highly compact or dense and has high heat resistance. It should be noted that the compactness or density of the paste is of such a degree or character that the sealing member 700 is capable of preventing the passage of ions therethrough. In addition, the high heat resistance is such as to make it possible for the sealing member 700 to withstand the high heating temperatures (e.g., 500° C. to 700° C.) associated with the heater 400.

The glass powder paste prepared as described above is applied over the outer peripheral surface of the leading end 321 of the center electrode 320 and the leading end 234 of the leading end portion 230 of the insulator 200 so as to assume or form a cross-sectional shape corresponding to that of a truncated cone, and is baked under predetermined operating (burning) conditions.

Thus, using the process outlined above, the gap 233 between the insulator 200 and the center electrode 320 is sealed with the sealing member 700. The discharge voltage generated at the electrode portion 122 of the outer electrode 120 and the leading end 321 of the center electrode 320 is affected, i.e., is reduced, only by soot, without being affected by the aforementioned particles contributing to electrical conductivity (conductive particles). In consequence, according to the soot sensor of this first embodiment, the soot can be detected with high accuracy without being affected by conductive particles.

It is noted that the soot sensitivity of the soot sensor of this first embodiment was measured in comparison with soot sensors of comparative examples not having the sealing portion of the present invention.

A GFG-1000 type soot generator (the amount of soot generated: 3 mg/m³) made by Palas GmbH of Germany was used in the aforementioned measurement. A measuring circuit was configured such that a high voltage from the aforementioned high-voltage circuit (not shown) was applied across the center electrode and the outer electrode, and the discharge voltage generated between the center electrode and the outer electrode was measured by an oscilloscope. The measurement was conducted 100 times for each soot sensor, and soot sensitivity was determined based on an average value of each measurement result.

Soot sensitivity is defined by the discharge voltage difference between (i) a discharge voltage occurring across the electrode portion 122 and the leading end 321 when there is no soot between the electrode portion 122 and the leading end 321 and (ii) the discharge voltage occurring across the electrode portion 122 and the leading end 321 when soot is present between the electrode portion 122 and the leading end 321.

According to the above-described measurement, the soot sensitivity of the soot sensor of the comparative example was 0V. In contrast, the soot sensitivity of the soot sensor of this first embodiment was 1,600 V. This occurred because, in the soot sensors of the comparative examples, there is a large effect due to ions according to a presumption which is described below.

When a high voltage is applied across the outer electrode 120 and the center electrode 320 of the soot sensor, the voltage generated between the outer electrode 120 and the center electrode 320 rises up to the aforementioned high voltage during a period of several tens of microseconds. During this voltage rise, the air in the atmosphere of the discharge portion 322 is dielectrically broken down and discharges. Such a discharge principally undergoes a transition to a Townsend discharge, to a corona discharge, and further to a spark discharge.

In the soot sensor described above, the heater 400 is connected to the metal shell 110 in the same way as the outer electrode 120. The resistance value of the heating element 430 of the heater 400 is typically several ohms or thereabouts. For this reason, it: is presumed or considered that the heater 400 is substantially at the same potential (ground potential) as the metal shell 110.

Accordingly, when a high voltage is applied across the outer electrode 120 and the center electrode 320, the predetermined high voltage is also applied across the heater 400 and the center electrode 320 through the insulator 200 and the gap 233 between this insulator 200 and the center electrode 320. For this reason, it is also presumed that a discharge takes place across the leading end portion 230 of the insulator 200 and the center electrode 320.

When such a discharge transitions to or becomes, for instance, a corona discharge, this corona discharge acts between the heating element 430 of the heater 400 and the center electrode 320 through a peripheral wall of the leading end portion 230 of the insulator 200. For this reason, it is presumed that the gas which is present between the leading end portion 230 of the insulator 200 and the center electrode 320 is ionized, i.e., produces ions.

It is presumed that the ions then move from the interior of the leading end portion 230 of the insulator 200 to the leading end 321 side of the center electrode 320, and electrically act as particles which contribute to electrical conductivity between the electrode portion 122 and the leading end 321 in the same way as soot.

This means that even if the atmosphere between the electrode portion 122 and the leading end 321 includes no soot and only includes ions, a discharge phenomenon is produced which is similar to the case where the atmosphere includes soot. In other words, the presence of soot is erroneously detected because of the presence of ions in the absence of soot, i.e., even though no soot is present. As a result, there is no difference in the discharge voltage regardless of the presence or absence of soot, and the soot detection accuracy is poor.

In contrast, with the soot sensor in accordance with this first embodiment, detection of soot with satisfactory accuracy is possible without being affected by conductive ions, as was presumed in the above discussion.

In this first embodiment configured as described above, the soot sensor is assumed in an important application to be mounted in an exhaust pipe of an automotive diesel engine so as to be exposed to the interior of the exhaust pipe.

If the detection output of the soot sensor of this first embodiment is used, fuel injection control of a diesel engine, for example, can be carried out with high accuracy, and the deterioration of a diesel particulate filter (DPF) for trapping particulate matter emitted from a diesel engine can also be accurately and properly detected. In addition, if the result of integration of concentrations of soot, which is the detection output of the soot sensor, is used, it is possible to estimate an appropriate timing for regeneration of the aforementioned DPF.

It is further noted that in this first embodiment, because the sealing member 700 is provided on the leading end 234 of the insulator 200 in such a manner as to cover the gap 233, it is possible to seal the gap 233 between the insulator 200 and the center electrode 320. Thus, according to the soot sensor of this first embodiment, soot can be detected with high accuracy without being affected by particles which contribute to electrical conductivity (conductive particles).

It is also noted that, in this first embodiment, because the sealing member 700 is formed of a glass, good heat resistance is provided in addition to compactness or density.

Accordingly, the sealing member 700 is capable of properly sealing the gap between the insulator 200 and the center electrode 320 even under the heating temperatures associated with the heating element 430.

In this first embodiment, the spacing or distance between a leading end 705 of the sealing member 700 and a leading end 435 (see FIG. 3) of the outer heating resistor portion 431 as measured along the outer surface 235 of the insulator 200 is 4 mm.

More generally, where the lower limit of the distance between the leading end 435 of the outer heating resistor portion 431 and the leading end 705 of the sealing member 700 along the outer surface 235 of the insulator 200 is set to 3 mm or more, the heating element 430 is not located too close to the leading end 321 of the center electrode 320. Accordingly, it is possible to prevent the heating element 430 from short-circuiting with the center electrode 320 or generating a discharge. Further, when the upper limit of the distance between the leading end 435 of the outer heating resistor portion 431 and the leading end 705 of the sealing member 700 along the outer surface 235 of the insulator 200 is set to 12 mm or less, it is possible to prevent the soot from becoming deposited on the insulator 200 and the sealing member 700.

In this embodiment, the insulator 200 preferably has a thickness of 1 mm at the position at which the heating element 430 is disposed. Because the insulator 200 thus has a thickness of not less than 0.7 mm at the position at which the heating element 430 is disposed, it is possible to prevent a discharge from taking place in the “thicknesswise” or transverse direction of the insulator 200, whereas otherwise the insulator 200 is otherwise too thin. In addition, because the insulator 200 has a thickness of not more than 3 mm at the position at which the heating element 430 is disposed, it is possible to prevent an increase in the heat capacity, whereas otherwise the insulator 200 is too thick.

### Second Embodiment

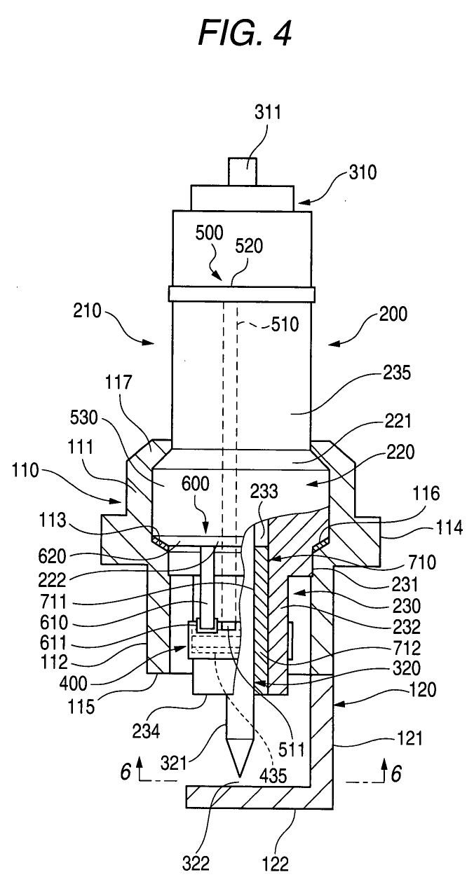

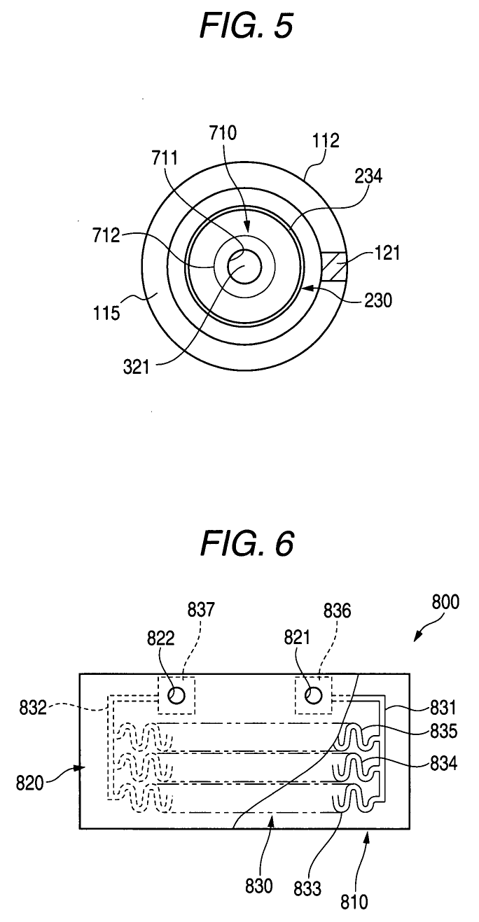

FIG. 4 shows a second embodiment of the spark plug type soot sensor in accordance with the invention. The soot sensor of this second embodiment has a configuration in which a cylindrical sealing member 710 is adopted, instead of the configuration of the sealing member 700 of the soot sensor in accordance with the above-described first embodiment.

The sealing member 710 is formed of a sealing material similar to that of the first embodiment into a cylindrical shape, and is fitted concentrically in the gap 233 between the center electrode 320 and the insulator 200.

As a result, the inner peripheral surface 711 and the outer peripheral surface 712 of the seal member 710 are in close, air-tight contact with the outer peripheral surface of the center electrode 320 and the inner peripheral surface of the insulator 200, respectively. The axial length of the sealing member 710 corresponds to the axial length of the leading end portion 230 of the insulator 200.

In this second embodiment, the sealing member 710 is formed as follows: the glass powder paste described in the first embodiment is filled in the gap 233 between the center electrode 320 and the cylindrical member 200, and is baked under the predetermined burning conditions described above in connection with the first embodiment.

In the second embodiment as thus configured, the sealing member 710 is fitted so as to be in close, air-tight contact with the outer peripheral surface of the center electrode 320 and the inner peripheral surface of the insulator 200. This sealing member 710 is formed so as to be closer to the leading end side of the sensor than the heating element 430.

For this reason, in the same way as described above for the above-described first embodiment, even if a discharge occurs between the heating element 430 and the center electrode 320, and ions are produced in the gap 233 at the leading end portion 230 of the insulator 200, these ions are suitably sealed within the gap 233 at the leading end portion 230 of the insulator 200 by the sealing member 710.

Accordingly, the aforementioned ions cannot move to the discharge portion 322. Consequently, in this second embodiment as well, the soot can be detected with high accuracy without being affected by the aforementioned ions, in the same way as described above for the above-described first embodiment.

In addition, in this second embodiment, because the sealing member 710 is formed of a glass, heat resistance is provided in addition to density or compactness. Accordingly, the sealing member 710 is capable of properly sealing the gap between the insulator 200 and the center electrode 320 even under the heating temperatures associated with the heating element 430.

In this second embodiment, the spacing or distance between the leading end 234 of the insulator 200 and the leading end 435 (see FIG. 3) of the outer heating resistor portion 431, as measured along the outer surface 235 of the insulator 200, is preferably set to 4 mm.

As discussed above, when the lower limit of the distance between the leading end 435 of the outer heating resistor portion 431 and the leading end 234 of the insulator 200 along the outer surface 235 of the insulator 200 is set to 3 mm or more, the heating element 430 is not located too close to the leading end 321 of the center electrode 320. Accordingly, it is possible to prevent the heating element 430 from short-circuiting with the center electrode 320 or generating a discharge. In addition, when the upper limit of the distance between the leading end 435 of the outer heating resistor portion 431 and the leading end 234 of the insulator 200 along the outer surface 235 of the insulator 200 is set to 12 mm or less, it is possible to prevent the soot from becoming deposited on the insulator 200.

In this embodiment, the insulator 200 preferably has a thickness of 1 mm at the position at which the heating element 430 is disposed. Because the insulator 200 thus has a thickness of not less than 0.7 mm at the position where the heating element 430 is disposed, it is possible to prevent a discharge from taking place in the “thicknesswise” or transverse direction of the insulator 200 whereas otherwise the insulator 200 is too thin and a discharge can occur. Further, because the insulator 200 has a thickness of not more than 3 mm at the position where the heating element 430 is disposed, it is possible to prevent an increase in heat capacity, whereas otherwise the insulator 200 is too thick and an increase in heat capacity can occur.

### Third Embodiment

FIG. 6 shows selected portions of a third embodiment of the invention. In this third embodiment, a heater 800 is employed or adopted instead of the heater 400 in accordance with the above-described first or second embodiment.

The heater 800 is used for effecting heat cleaning as described for the above-described first or second embodiment.

In the same way as the heater 400, heater 800 is attached to, i.e., extends over, the entire periphery of the small-diameter portion 232 of the leading end portion 230 of the insulator 200 described in the above-described first or second embodiment.

As shown in FIG. 6, heater 800 includes two alumina sheets 810 and 820 and a heating element 830. The heating element 830 has two lead portions 831 and 832, three heating resistor portions 833, 834, and 835, and both positive and negative “both side” electrode pads 836 and 837 (see FIG. 6).

The three heating resistor portions 833, 834, and 835 extend parallel to each other along an inner surface of alumina sheet 810 between the both lead portions 831 and 832, and heating resistor portions 833, 834, and 835 are connected at both ends to the both lead portions 831 and 832. It should be noted that, in this third embodiment, the respective heating resistor portions 833, 834, and 835 are formed with a corrugated pattern having alternately arranged upper projecting portions and lower projecting portions, as shown in FIG. 6.

The positive and negative “both side” electrode pads 836 and 837 are formed on the inner surface of the alumina sheet 810 through respective opposing ends of the both lead portions 831 and 832.

The alumina sheet 820 is pressure-bonded to the inner surface of the alumina sheet 810 with the heating element 830 placed therebetween. Through holes 821 and 822 are formed in this alumina sheet 820 at positions corresponding to respective central portions of the both electrode pads 836 and 837.

### Fourth Embodiment

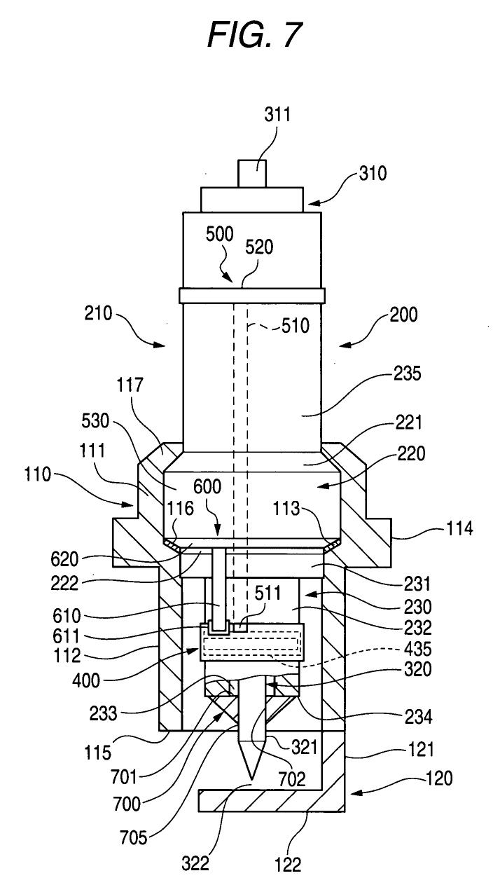

FIG. 7 shows a fourth embodiment of the invention. In this fourth embodiment, the metal shell 110 described in the first embodiment is configured as described below.

As shown in FIG. 7, the leading end 705 of the sealing member 700 is located rearwardly of the leading end 115 of the metal shell 110. Further, the leading end portion 112 of the metal shell 110 is provided in such a manner as to, i.e., is configured so as to, surround the leading end portion 230 of the insulator 200.

With this construction, the leading end portion 230 of the insulator 200, together with the sealing member 700, is located on the inner side of the metal shell 110. Accordingly, it is difficult for the soot to move around into the metal shell 110, and it is unlikely that the leading end portion 230 of the insulator 200 and the sealing member 700 will be exposed to a significant amount of soot. Thus, the leading end portion 230 of the insulator 200, together with the sealing member 700, can be isolated from the soot.

### Fifth Embodiment

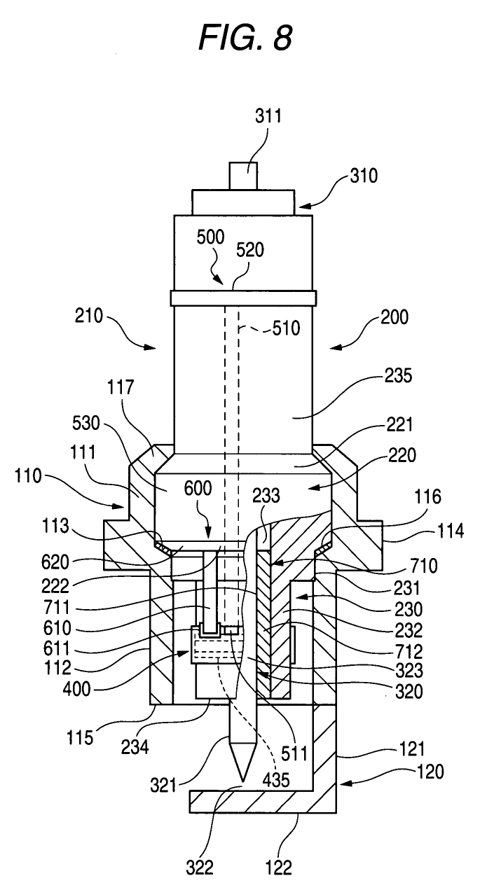

FIG. 8 shows a fifth embodiment of the invention. In this fifth embodiment, the metal shell 110 described in the first embodiment is configured as described below.

As shown in FIG. 8, the leading end 234 of the insulator 200 is located rearwardly of the leading end 115 of the metal shell 110. Further, the leading end portion 112 of the metal shell 110 is provided in such a manner as to surround, i.e., is configured so as to surround, the leading end portion 230 of the insulator 200.

With this construction, the leading end portion 230 of the insulator 200 is located on the inner side of the metal shell 110. Accordingly, it is difficult for the soot to move around into the metal shell 110, and the soot is effectively prevented from being deposited on the leading end portion 230 of the insulator 200. Thus, the leading end portion 230 of the insulator 200 can be isolated from the soot.

It should be noted that the invention in its implementation is not limited to the above-described embodiments, and, for example, the following various modifications can be made therein.

First, the material used in forming the sealing member 700 or 710 is required to have high density or compactness and high heat resistance in order to provide the aforementioned sealing of ions into the insulator 200 and the aforementioned heat resistance against the heating temperatures (e.g., 500° C. to 700° C.) associated with the heaters 400 and 800. However, the material used in forming the sealing member 700 or 710 is not limited to the materials described in the foregoing embodiments, and any material, insofar as it satisfies these requirements, may be used as the material of the sealing member. For example, a ceramic may be used as the material for forming the sealing member.

Further, a metal may be employed as the material used in forming the sealing member 700 in the above-described first embodiment. It should be noted, however, that the spacing or distance between the leading end 435 of the heating element 430 and the leading end 234 of the insulator 200, along the outer surface 235 of the insulator 200, is, as stated above, preferably not less than 3 mm and not more than 12 mm. Thus, because the lower limit of the length between the leading end 435 of the heating element 430 and the leading end 234 of the insulator 200 along the outer surface 235 is set to 3 mm or more, the heating element 430 is not located too close to the sealing member 700. Accordingly, it is possible to prevent the heating element 430 from short-circuiting with the sealing member 700 or generating a discharge. Further, because the upper limit of the distance between the leading end 435 of the heating element 430 and the leading end 234 of the insulator 200 along the outer surface 235 is set to 12 mm or less, it is possible to prevent the soot from becoming deposited on the insulator 200.

In addition, in the above-described first embodiment, in the modification wherein a metal is used as the material for forming the sealing member 700, the leading end 234 of the insulator 200 is preferably located closer to the rear end side of the sensor device than the leading end 115 of the metal shell 110. Because the leading end 234 of the insulator 200 is located closer to the rear end side than the leading end 115 of the metal shell 110, it is difficult for the soot to be applied to the insulator 200 from outside of the metal shell 110, thereby making it possible to prevent the soot from being deposited on the insulator 200.

It should be noted that if the heating temperatures associated with the heater 400 and 800 is not high, a resin may be used as the material for forming the sealing member 700 or 710.

In another modification, the shape of each heating resistor portion of the heater is not limited to the pattern of each heating resistor portion of the heater 400 or 800, and may be altered, as desired or required.

In a further modification, the heater 400 or 800 may not be attached to, i.e., may not cover, the entire periphery of the leading end portion 230 of the cylindrical member 200, but may be arranged to be attached to or cover only a portion of that entire periphery.

In yet another modification, an arrangement may be provided wherein the discharge portion is formed between the center electrode and the inner wall of a pipe where the soot sensor is disposed, and the outer electrode may not be used or may be dispensed with.

This application is based on Japanese Patent Application JP 2006-182915, filed Jul. 3, 2006, and Japanese Patent Application JP 2007-123035, filed May 8, 2007, the entire content of both of which is hereby incorporated by reference, the same as if this content were set forth at length.

Although the invention has been described above in relation to preferred embodiments and modifications thereof, it will be understood by those skilled in the art that other variations and modifications can be effected in these preferred embodiments without departing from the scope and spirit of the invention.

What is claimed is:

1. A soot sensor comprising:
a center electrode extending in an axial direction;
a cylindrical insulator, provided around a periphery of the center electrode, from which a leading end of the center electrode protrudes, the insulator including a heating element; and
a sealing member sealing a gap between the insulator and the center electrode.

2. The soot sensor as claimed in claim 1, wherein the sealing member is provided on a leading end of the insulator so as to cover the gap.

3. The soot sensor as claimed in claim 2, wherein the sealing member comprises at least one of a glass and a ceramic.

4. The soot sensor as claimed in claim 3, wherein a leading end of the heating element and a leading end of the sealing member, are spaced apart along an outer surface of the insulator by a distance of between 3 mm and 12 mm.

5. The soot sensor as claimed in claim 3, further comprising: a hollow metal shell provided around a periphery of the insulator, wherein a leading end of the sealing member is located closer to a rear end side of the sensor than the leading end of the metal shell.

6. The soot sensor as claimed in claim 2, wherein the sealing member comprises a metal.

7. The soot sensor as claimed in claim 1, wherein the sealing member is provided in the gap at a position closer to a leading end side of the sensor than at least the heating element.

8. The soot sensor as claimed in claim 7, wherein the sealing member comprises at least one of a glass, a ceramic, and a metal.

9. The soot sensor as claimed in claim 6, wherein a leading end of the heating element and a leading end of the insulator are spaced apart by a distance of between 3 mm and 12 mm along an outer surface of the insulator.

10. The soot sensor as claimed in claim 6, further comprising: a hollow metal shell provided around a periphery of the insulator, wherein a leading end of the insulator is located closer to a rear end side of the sensor than a leading end of the metal shell.

11. The soot sensor as claimed in claim 1, wherein the center electrode comprises a positive side electrode.

12. The soot sensor as claimed in claim 1, wherein the insulator has a thickness of between 0.7 mm and 3 mm at a position at which the heating element is disposed.
